(19) United States
(12) Patent Application Publication
(10) Pub. No.: US 2013/0149253 A1
(43) Pub. Date: Jun. 13, 2013

(54) ORAL DOSAGE FORM OF PREGABALIN

(71) Applicants: Dominique Meergans, Munich (DE); Jana Paets, Bonn (DE)

(72) Inventors: Dominique Meergans, Munich (DE); Jana Paets, Bonn (DE)

(21) Appl. No.: 13/812,506

(22) PCT Filed: Aug. 2, 2011
(86) PCT No.: PCT/EP2011/003873

§ 371 Date: Mar. 4, 2013

(30) Foreign Application Priority Data
Aug. 3, 2010 (EP) 10 008 104.1

(51) Int. Cl. A61K 31/197

(52) U.S. Cl. CPC A61K 31/197
USPC 424/44; 514/561

(57) ABSTRACT

The invention relates to oral dosage forms of pregabalin, preferably for the modified release, and to processes for producing it.

The invention relates to oral dosage forms of pregabalin, preferably for the modified release, and to processes for producing it.

The IUPAC name of pregabalin [INN] is (S)-3-(aminomethyl)-5-methyl hexanoic acid. The chemical structure of pregabalin is shown in formula (1) below:

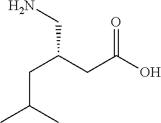

Pregabalin is an analogue of the physiologically important endogenous neurotransmitter γ-amino butyric acid (GABA), which is involved in the regulation of neural processes. It binds to α2δ sub-units of calcium channels. The synthesis of pregabalin and its use as a drug with anticonvulsive effect are described in EP 0 641 330.

Pregabalin is currently approved in Europe for the treatment of epilepsy, neuropathological pain and generalised anxiety disorders. Among the possible causes of neuropathological pain may be: diabetic polyneuropathy, post-zoster neuralgia, tumours, chemotherapy, trigeminal neuralgia, alcohol abuse, vitamin B deficiency, phantom pain, borrelia infection, complex regional pain syndrome, carpal tunnel syndrome, back pain and AIDS. In the USA, pregabalin is also approved for the treatment of fibromyalgia.

Pregabalin is marketed under the trade name Lyrica® in the form of immediate-release tablets. Standard therapeutic doses are 150 mg to 600 mg daily, divided into two or three individual doses.

In order to increase compliance in patients and to achieve plasma concentrations of the active agent which are as constant as possible, a dosage form is desirable which can be taken once daily.

The development of a suitable formulation of this kind is, however, rendered more difficult by the fact that pregabalin cannot be absorbed in the entire gastrointestinal tract (GIT). The active agent is only absorbed in the upper sections of the gut. This means that the introduction of delayed release from the dosage form would result in large amounts of active agent being carried past the absorption window during passage through the gut. It is therefore desirable to have a dosage form which has a longer dwell time in the upper GIT and which, during that time, releases the active agent continuously over a sufficiently long period.

The international patent application WO 2007/052125 proposes gastroretentive formulations for the controlled release of pregabalin. The release of pregabalin is controlled by a matrix formulation of polyvinyl acetate and polyvinyl pyrrolidone. Gastroretention is achieved by an immediate increase in the size of the dosage form in the gastric environment, so that, because of its size, the formulation is no longer able to pass the pylorus. In addition, it is disclosed that the formulation can advantageously be taken together with the intake of food, resulting in a longer dwell time in the stomach. In addition, the dosage form is also particularly suitable at night, since stomach activity is usually reduced at that time.

The substances used in this formulation swell rapidly to a considerable extent. This entails the risk of premature swelling in the oesophagus, which can cause it to become blocked. In addition, it would be desirable to have a formulation which could be taken independently of the intake of food and/or the time of day.

It was therefore an object of the present invention to provide a formulation for the modified release of pregabalin which does not entail the risk of premature swelling in the oesophagus, or only to a substantially lesser extent. It is a further, or alternative, object of the present invention to provide a formulation which is not dependent on reduced stomach activity (e.g. after the intake of food or at night). The dosage form should have a superior bioavailability, even after storage.

The objects are achieved by the gastroretentive principle on which the oral dosage form of the invention is based.

One subject matter of the present invention is an oral dosage form, preferably a tablet, preferably for the modified release of pregabalin, comprising
(a) pregabalin
in a matrix comprising
(b) a swelling agent,
(c) a matrix former and
(d) a buoyancy agent or a sedimentation agent.

The dosage form of the invention is based on the concept of providing a gastroretentive dosage form which not only swells in the stomach, but either floats in the stomach or sinks in the stomach. These two phenomena prevent excessively rapid passage through the gastric tract, and do so independently of the degree of swelling.

The use of a buoyancy agent or sedimentation agent thus makes it possible to use a swelling agent which swells less powerfully or considerably more slowly, so that the risk of occlusion of the oesophagus can be eliminated or at least substantially reduced.

Furthermore, the buoyancy or sinking at least reduces dependency on the intake of food or on sleep.

In addition, the formulation comprising components (a) to (d) enables the compression of tablets having superior friability properties. Further, the formulation shows superior flowability properties so that the compression process could be improved. Preferably, an essentially pH independent release profile can be achieved.

The oral dosage form is preferably designed such that after 10 minutes of swelling in deionised water at 37° C., a volume of the oral dosage form is no more than 25% greater than a volume of the dosage form before swelling, preferably no more than 20%, no more than 15% or no more than 12.5%. The volume of the dosage form before swelling expresses the volume of the dosage form in a dry state at room temperature and normal pressure and in this respect forms the reference point for the increase in volume.

Since oral dosage forms of the present kind are usually taken with liquid, for which water is usually chosen, the swelling behaviour in water is relevant in this connection. The increase in volume is, however, preferably also within the given range even when other liquids are used, such as when taken with juice or other liquids which have a pH different from water. As one example of these scenarios, therefore, the increase in volume in hydrochloric acid in the pH range from 1 to 7 is preferably within the numerical range specified above for water, and preferably also in sodium hydroxide solution in the range from pH 10 to 7.

“Deionised water” in the present context means water with a conductivity at 25° C. of 5-10⁻⁶ S/m or less.

The oral dosage form contains a swelling agent, which is preferably capable of swelling in gastric juice.

It is preferable in this connection that after 45 minutes of swelling in 0.1 N hydrochloric acid at 37° C., a volume of the oral dosage form is at least 7.5% greater than a volume of the dosage form before swelling, preferably at least 9%, at least 10%, at least 12.5% or at least 15%. It is also preferable that after 120 minutes of swelling in 0.1 N hydrochloric acid at 37° C., a volume of the oral dosage form is at least 10% greater than a volume of the dosage form before swelling, preferably at least 12.5%, at least 15%, at least 17.5%, or at least 20%.

The swelling volume is an indicator of the swelling behaviour in the stomach. The more strongly the oral dosage form is capable of swelling, the smaller the oral dosage form can be in the dry state, which is advantageous for the patients with regard to the swallowing behaviour.

It is preferable that the maximum swelling volume is specifically not reached within 10, 20 or 60 minutes of swelling in the above-mentioned HCl medium.

The purpose of the buoyancy agent is to provide the oral dosage form with buoyancy so that it floats on the surface of the gastric juice or in the upper region of the stomach. This can be achieved for example in that after contact with the stomach liquid, the oral dosage form has a specific density which is lower than that of the stomach liquid. The low specific density can be achieved via, for example, the swelling behaviour, or through the formation of gas within the dosage form, and in particular within the matrix.

Buoyancy is tested using 0.1 N hydrochloric acid. In 0.1 N hydrochloric acid with a volume of 1,000 mL at 37° C. in a beaker of 2,000 mL volume, the oral dosage form rises from the bottom (with unmoving liquid, i.e. without stirring), particularly preferably after no more than 30 minutes, more preferably no more than 20 minutes and even more preferably no more than 10 or 5 or 3 minutes.

A buoyancy agent is preferable which is able, upon contact with 0.1 N hydrochloric acid, to release carbon dioxide and/or nitrogen. Ideally, the buoyancy agent is suitable, upon contact with hydrochloric acid in the pH range from 0 to 6.5, for releasing carbon dioxide and/or nitrogen.

Conceivable buoyancy agents based on the function of gas formation are generally the disintegrants usually employed in the field for effervescent tablets which develop gas. Conceivable buoyancy agents releasing carbon dioxide are in particular pharmaceutically acceptable carbonates and hydrogen carbonates, especially those of the alkali metals, and mixtures thereof. Examples are sodium hydrogen carbonate, potassium hydrogen carbonate, potassium carbonate, calcium hydrogen carbonate and magnesium hydrogen carbonate. Sodium hydrogen carbonate is particularly preferable in this connection.

Other suitable buoyancy agents are sodium glycine carbonate, an addition compound of sodium hydrogen carbonate and glycine, and arginine carbonate, the corresponding addition compound of arginine.

Possible buoyancy agents can also further (additionally) comprise ascorbic acid, tartaric acid, citric acid, their pharmaceutically acceptable salts and combinations thereof.

In addition, suitable combinations of the above-mentioned further buoyancy agents can also be used. Examples for said combinations are hydrogen carbonate/ascorbic acid, hydrogen carbonate/tartaric acid, hydrogen carbonate/citric acid, carbonate/tartaric acid, carbonate/citric acid and/or carbonate/ascorbic acid.

Buoyancy agent(s) is/are usually contained in an amount of 1 to 20% by weight, preferably 3 to 15% by weight of the total weight of the oral dosage form, such as in amounts of at least 3.5% by weight, at least 4% by weight, at least 5% by weight or at least 6% by weight and/or, for example, up to a maximum of 14% by weight, a maximum of 13% by weight or a maximum of 10% by weight. If the amount of buoyancy agent is too high, this can jeopardise the cohesion of the matrix and hence the function of the oral dosage form.

As an alternative to the buoyancy agent, a sedimentation agent can be used. A sedimentation agent of this kind preferably serves to make the swollen oral dosage form heavier, so that it sinks, ideally right down to the bottom of the stomach.

In a preferred embodiment, the sedimentation agent has a density of 1.5 to 8.0, more preferably of 1.8 to 6.0, still more preferably of 1.9 to 5.0, particular preferred of 2.0 to 4.0 and especially of 2.1 to 3.5 g/cm³. Preferably, the density is measured according to Ph. Eur. 7.0, 2.9.23, in particular at 20° C. Preferably a Micromeritics AccuPyc 1340 is used. Further, preferably the sedimentation agent is not able, upon contact with acid, to release gas, e.g. carbon dioxide or nitrogen. Further, the sedimentation agent preferably does not comprise acidic groups

Conceivable sedimentation agents are, for example, pharmaceutically acceptable inorganic salts, such as chlorides, sulphates, hydrogen phosphates, phosphates, oxides and the like. Examples of these would be sodium chloride, calcium chloride, sodium sulphate, calcium sulphate, sodium phosphate, calcium phosphate, calcium hydrogen phosphate, barium sulphate, titanium dioxide, zinc oxide or iron powder. It is particularly preferable to use sodium chloride. Combinations of sedimentation agents may also be used. The oral dosage form preferably usually contains sedimentation agents in an amount of 1 to 20% by weight, preferably, 3 to 15% by weight of the total weight, such as in amounts of at least 3.5% by weight, at least 4% by weight, at least 5% by weight or at least 6% by weight and/or, for example, up to a maximum of 14% by weight, a maximum of 13% by weight or a maximum of 10% by weight.

While embodiments containing both buoyancy agents and sedimentation agents are conceivable, they would not appear to make much technical sense unless they were both contained in the matrix in such forms that they developed their effect with a time gap. Sedimentation agents and buoyancy agents are usually employed as alternatives.

The matrix former is generally used to provide a matrix structure which provides the oral dosage form and in particular the matrix with physical stability during the desired dwell time in the stomach, or holds the matrix components together mechanically.

The principal purpose of the swelling agent is to increase the volume of the oral dosage form by swelling in contact with the gastric juice.

In sample embodiments, the swelling agent and matrix former may be the same substance or the same mixture of substances. To put it another way, one substance can provide both functions, so that it is then a swellable matrix former.

In preferred embodiments, however, the swelling agent and matrix former are different substances or different mixtures of substances. Then the two functions are provided by different substances. This may, for example, be the case if the matrix former is not or only poorly swellable. In this context, the difference may relate to the substance itself, such as the chemical composition, as may be seen from the representation of a formula for example. In the case of polymers, for instance, the substances may, however, also differ with regard to such properties as their molecular weight, density, viscosity in solution or the degree of cross-linking. They are preferably substances which are clearly distinct, such as with regard to their sum formulae or their structure or physical properties.

The matrix former may, for example, be a hydrophilic matrix former. Examples of conceivable matrix formers are generally polymers, oligomers and natural substances. Substances from the group of polysaccharides or alginates may be used as matrix formers. Substances from the groups of starches or also cellulose derivatives would also be suitable for this purpose. The matrix former may comprise or consist of one or more substances from these classes of substances. Examples of these would be:

polyvinyl acetate, polyethylene glycol, polyvinyl alcohol, cellulose and their ethers and esters, such as cellulose powder, microcrystalline cellulose, ethyl cellulose, carboxymethyl cellulose, hydroxyethyl cellulose, hydroxypropyl cellulose, gum arabic, carrageenan, gelatine, gum traganth, amylose, maltodextrins, polysaccharides such as guar gum or alginates, starch, modified starch, pectins, sugar alcohols or combinations or copolymers thereof. Polyvinyl acetate is preferable, such as in the form of the commercially available Kollidon®, particularly preferably Kollidon® SR. According to the manufacturer's statements, Kollidon® SR is a mixture of 80% polyvinyl acetate and 19% povidone (polyvinyl pyrrolidone) and 0.8% sodium lauryl sulphate and 0.6% silica as stabilisers.

However, the matrix former preferably does not contain and is not a polyacrylate or other polymer based on a derivative of acrylic acid or methacrylic acid.

In polymeric matrix formers, the weight-average molecular weight is preferably in the range from 1×10³ to 1×10⁷ g/mol, especially at least 2.5×10³ g/mol. It is further preferable that the viscosity of a 2% (w/w) aqueous solution of the matrix former at 25° C. should be in the range from 30 to 10,000 mPa·s, such as in the range from at least 2,000 or 4,500 mPa·s.

The matrix former is preferably contained in an amount of 5 to 45% by weight of the oral dosage form, such as in an amount of 15 to 40% by weight or 20 to 35% by weight.

The swelling agent may comprise or consist of one or more of the following substances:

polyethylene oxide or polyethylene glycol, preferably non-ionogenic polyethylene glycols; cellulose derivatives such as cellulose esters or ethers, e.g. hydroxyalkyl cellulose such as hydroxyethyl cellulose, hydroxypropyl cellulose, hydroxypropyl methyl cellulose, carboxymethyl cellulose, methyl cellulose; polyvinyl alcohol; polyvinyl pyrrolidone; carrageenan; pectins; alginates; colloidal magnesium/aluminium silicates.

Water-soluble resins based on non-ionogenic polyethylene glycols are particularly preferable. The viscosity of a 2% (w/w) aqueous solution of the preferably non-ionogenic polyethylene glycols at 25° C. is preferably in the range from 1,000 mPa·s, more preferably 2,000 to 8,000 mPa·s, even more preferably 2,000 to 4,000 mPa·s. They may, for example, have a weight-average molecular weight in the range from about 10⁵ to 5×10⁶ g/mol. Polyethylene glycols of this kind are, for example, obtainable under the trade name Polyox®, the products with the trade name Polyox® WSR N60K being particularly preferable.

In the polymeric swelling agents in general, the weight-average molecular weight is preferably in the range from 10³ to 1.5×10⁶ g/mol, especially at least 10⁴ g/mol. It is further preferable that the viscosity of a 2% (w/w) aqueous solution of the swelling agent at 25° C. should be in the range from 10,000 to 80,000 mPa·s, such as in the range from at least 15,000 mPa·s or at least 20,000 mPa·s.

The swelling agent may further be water-soluble or water-insoluble. “Water-insoluble” means a solubility of less than 33 mg/ml in deionised water, whereas everything which is higher than that is referred to herein as water-soluble, i.e. everything which is not water-insoluble.

The swelling agent is preferably contained in an amount of 10 to 60% by weight of the oral dosage form, such as in an amount of 15 to 60% by weight or 20 to 60% by weight.

The swelling agent itself preferably has a low or moderate swelling rate. Swelling agents or mixtures of swelling agents are preferable whose volume, after 10 minutes of swelling in deionised water at 37° C. does not amount to more than 105%, preferably not more than 110%, for example not more than 120% or 125% compared to the dry swelling agent before the swelling process in water.

The oral dosage form is preferably a tablet, especially a matrix tablet, preferably with a size of at least 9 mm, such as at least 10 mm. A greatest dimension of the oral dosage form in cross-section, e.g. the matrix tablet, after 30 minutes of swelling in 0.1 M hydrochloric acid at 37° C. is preferably 9 mm or more, such as at least 10, at least 11, at least 12 or at least 13 mm. The greatest dimension in cross-section (linear distance) refers in the case of a round tablet to the diameter, for example (not the circumference), and in the case of a caplet (a capsule-shaped tablet) to the length etc. A greatest dimension of the oral dosage form in cross-section after 60 minutes of swelling in 0.1 M hydrochloric acid at 37° C. is preferably at least 10, at least 11, at least 12 or at least 13 mm.

As a measure of the swelling capacity, the swelling index is determined. For this purpose, the increase in size of the oral dosage form, e.g. the tablet, is measured after it has been placed in a 2,000 mL beaker in 1 litre 37° C. warm 0.06 N hydrochloric acid. The oral dosage form is removed at regular intervals and its dimension measured with a sliding caliper. In the case of oral dosage forms which are not rotationally symmetrical, the greatest (linear) dimension is taken as the basis and measured, such as the length in the case of caplets. In the case of round (usually disk-shaped) tablets, their diameter is determined.

In addition to this, swelling agents and matrix formers, alone or in combination, preferably cause the modified, particularly preferably extended, release of the pregabalin from the oral dosage form. Embodiments are, however, also encompassed in which the oral dosage form contains further ingredients that influence the release of the pregabalin (perhaps additionally). On the one hand, further ingredients may be added to the matrix which influence or control the release behaviour. It is also conceivable for pregabalin to be used in a form that is only slowly soluble or the like.

Oral dosage forms are preferable which have a release behaviour, measured with USP Test Apparatus II using 900 ml 0.06 N HCl at pH 1.4 at a speed of 50 revolutions per minute and at a temperature of 37° C., which satisfies at least one, preferably two, more preferably three or most preferably all of the following conditions:

| Time [minutes] | % pregabalin released |
|---|---|
| 30 | 5-20 |
| 60 | 10-25 |
| 90 | 15-30 |
| 120 | 20-40 |
| 320 | 30-60 |
| 560 | 50-80 |
| 720 | 60-90 |

Apart from the above-mentioned ingredients, the oral dosage form may, for example, contain one or more additional ingredients, such as a gelling agent, e.g. to control (or, where applicable, to assist in controlling) the release. Conceivable gelling agents are, for example, pharmaceutically acceptable hydrogel or hydrocolloid forming agents, such as bentonite, gum traganth, xanthan gum, cellulose derivatives such as cellulose ethers or esters, e.g. methyl cellulose, hydroxyalkyl cellulose, especially hydroxyethyl cellulose, carrageenans, polysaccharides, guar gum, ceratonia and particularly preferably polyacrylic acid, such as carbomers (carbopols). Carbopols are (homo- or co-)polymers of acrylic acid with a high relative molar mass, which are cross-linked with polyalkene ethers of sugars or polyalcohols. Those carbomers may, for example, be used in which the polyacrylic acid (homopolymer) is cross-linked with allyl sucrose or allyl pentaerythritol. Carbopol® 71G is preferable.

It is also possible to use mixtures of gelling agents. When reference is made herein to a gelling agent, it is regarded as different from a matrix former and swelling agent, i.e. as an additional excipient. That does not mean that matrix formers and/or swelling agents could not have any gel-forming properties.

The viscosity of a 2% (w/w) aqueous solution of the gelling agent at 25° C. is in the range from 200 to 45,000 mPa·s, such as in the range from at least 10,000 or at least 16,000 mPa·s.

The gelling agent, if present, is preferably contained in an amount of 0 to 25% by weight of the oral dosage form, such as in an amount of 5 to 25% by weight or 5 to 20% by weight.

As a matter of principle, the term “pregabalin” in the context of this application comprises both the “free amino acid” described at the beginning (which is present in the form of a zwitterion) and also pharmaceutically acceptable salts, solvates, complexes and polymorphs thereof. Furthermore, pregabalin may also be used as the racemate, but preferably as an enantiomer. These may be one or more salts, which may also be present in a mixture. Examples to be mentioned here are the acid addition salts of inorganic and/or organic acids, e.g. hydrochlorides, carbonates, hydrogen carbonates, acetates, lactates, butyrates, propionates, sulphates, methane sulphonates, citrates, tartrates, nitrates, sulphonates, oxalates and/or succinates; and also base addition salts such as those of the alkali or alkaline earth metal cations or amines.

The pregabalin used is normally crystalline material, though it can also be used in amorphous or partially amorphous form. Statements regarding the amount of pregabalin herein refer to the free amino acid excluding any proportion of hydrate or solvate that might be present.

The oral dosage form of the invention contains, for example, 10 to 1,000 mg pregabalin, such as between 50 and 600 mg pregabalin. Pregabalin accounts, for example, for an amount of 2% by weight to 50% by weight of the oral dosage form.

In addition to the above-mentioned ingredients, the oral dosage form may also comprise further pharmaceutically acceptable excipients, such as flow-regulating agents. Flow-regulating agents are particularly preferable if the oral dosage form is present in the form of a tablet. Their task is to reduce both the interparticular friction (cohesion) between the individual particles in a tableting mixture and their adherence to the wall surfaces of the press mould (adhesion). One example of an additive to improve the powder flowability is disperse or colloidal silica (e.g. Aerosil®). Preferably, silica is used with a specific surface area of 50 to 400 m²/g, determined by gas adsorption in accordance with Ph. Eur., 6th edition 2.9.26.

In a further embodiment, the oral dosage form, especially when present in tablet form, may, for example, additionally contain lubricants. Lubricants are generally used in order to reduce sliding friction. In particular, the intention is to reduce the sliding friction found during tablet pressing between the punches moving up and down in the die and the die wall, on the one hand, and between the edge of the tablet and the die wall, on the other hand. Suitable lubricants are, for example, stearic acid, adipic acid, sodium stearyl fumarate, (Pruv®), magnesium stearate, calcium stearate or mixtures thereof.

In addition, the oral dosage form may contain a wetting agent. The task of wetting agents is to improve the wettability of active agents and/or excipients. Examples of wetting agents are anionic, cationic, amphoteric or non-ionic surfactants. It may, for example, be possible to use the following surfactants such as representatives of the following classes of surfactants: polyoxyethylene fatty alcohol ether, e.g. macrogol lauryl ether, (e.g. Brij®), ethoxylated sorbitan fatty acid ester (also known as polyoxyethylene sorbitan fatty acid ester, e.g. Tween®), polyoxyethylene fatty acid glycerides, polyoxyethylene fatty acid esters, e.g. macrogol stearate 400, sucrose fatty acid esters, non-ionic macromolecular surfactants, such as poloxamers, sodium lauryl sulphate (also known as sodium dodecyl sulphate), sodium cetyl stearyl sulphate, phospholipids, ethoxylated castor oil, soya lecithin and others, and also mixtures of two or more of the above-mentioned surfactants.

The oral dosage form may contain the above-mentioned optional pharmaceutically acceptable excipients or may be free of them.

Under a further aspect, the invention relates generally to an oral dosage form for the modified release of pregabalin, comprising pregabalin in a swellable matrix, wherein a volume of the oral dosage form, after 10 minutes of swelling in deionised water at 37° C. is no more than 30% larger than a volume of the dosage form before swelling and wherein a volume of the oral dosage form after 45 minutes of swelling in 0.1 M hydrochloric acid at 37° C. is at least 30% larger than a volume of the dosage form before swelling.

In preferred embodiments, the oral dosage form of the invention is suitable for administration once daily.

In this context, the oral dosage form of the invention is preferably used for the prevention and/or treatment, particularly preferably the treatment, of a disease or complaints responsive to pregabalin. The disease is, or the complaints may be, epilepsy, neuropathological pain, generalised anxiety disorders and/or fibromyalgia. The prevention and/or treatment of neuropathological pain relates, for example, to neuropathological pain in connection with diabetic polyneuropathy, post-zoster neuralgia, tumours, chemotherapy, trigeminal neuralgia, alcohol abuse, vitamin B deficiency, phantom pain, borrelia infection, complex regional pain syndrome, carpal tunnel syndromes, back pain and AIDS. In addition, the prevention and/or treatment of restless-leg syndrome, bipolar disorder, migraine and withdrawal symptoms are conceivable.

According to the invention, oral dosage forms, especially matrix tablets, preferably have a mass of 150 to 2,000 mg, preferably 200 to 1,000 mg, or particularly preferably 250 to 800 mg.

In the context of the invention, the resulting tablets may be coated or uncoated (film-coated or non-film-coated). The film formers used for the coating process may, for example, be cellulose derivatives, such as methyl cellulose (MC), ethyl cellulose (EC), hydroxyethyl cellulose (HEC), methacrylic acid/acrylate copolymers, such as methacrylic acid/ethacrylate copolymer or methacrylic acid/methyl methacrylate copolymer, vinyl polymers, such as polyvinyl pyrrolidone or polyvinyl acetate phthalate or natural film formers, such as shellack. The thickness of the layer, where present, is usually 0.1 to 100 μm, preferably 1 to 80 μm.

The oral dosage forms of the invention are preferably provided without film-coating or other coating.

The structure or the retentive principle of the oral dosage forms of the invention, especially tablets, comprising the above-mentioned ingredients, makes it possible to achieve advantageous hardness. In particular, even when a comparatively lower compression pressure is employed in compression, greater hardness can be achieved than in the state of the art. Exemplary embodiments have a hardness of 50 to 250 N, particularly preferably at least 100 to 230 N, especially at least 150 N. The hardness is determined in accordance with Ph. Eur. 6.0, section 2.9.8.

In addition, the resulting tablets preferably have a lower friability, such as a friability of 0.1 to 0.8%, preferably 0.2 to 0.6% and particularly preferably 0.3 to 0.5%. The friability is determined in accordance with Ph. Eur. 6.0, section 2.9.7.

In addition, one subject matter of the present invention is a process for producing the oral dosage form of the invention, comprising one of the following processes:
(a) mixing pregabalin, matrix former, swelling agent and optionally one or more pharmaceutically acceptable excipients and subsequently compressing the resulting mixture into a tablet,
(b) granulating pregabalin, matrix former, swelling agent and optionally one or more pharmaceutically acceptable excipients into granules, optionally adding one or more pharmaceutically acceptable excipients to the granules and subsequently compressing them into a tablet,

Regarding the processes mentioned above, the statements concerning the oral dosage form apply analogously.

Process (a), direct tableting, is preferable.

“Granulating” is generally understood to mean the formation of relatively coarse or granular aggregate material as a powder by assembling and/or aggregating finer powder particles (agglomerate formation, or build-up granulation) and/or the formation of finer granules by breaking up coarser aggregates (disintegration, or break-down granulation).

Granulation can conventionally mean wet or dry granulation. Dry granulation is generally carried out using pressure or temperature. Wet granulation (moist granulation) is generally carried out using surface stabilisers and/or solvents or dispersants. Granulation is generally carried out in conventional granulating devices, such as extruder, perforated-disk, perforated-roll or fluidised-bed granulators. Compulsory mixers or spray dryers can likewise be used. The granulating can generally be performed with processes known in the state of the art. If wet granulation is performed, a “drying” step is usually employed. The drying step can be performed after or at the same time as the granulation step. “Drying” for the purposes of this invention is understood to mean the separation of liquids adhering to solids. Drying generally takes place in conventional drying equipment, such as cabinet or tray dryers, vacuum dryers, fluidised bed dryers, spray dryers or freeze dryers. The drying and granulation process is preferably performed in one and the same apparatus.

The invention will now be explained in more detail on the basis of the following examples with reference to the Figures. There,

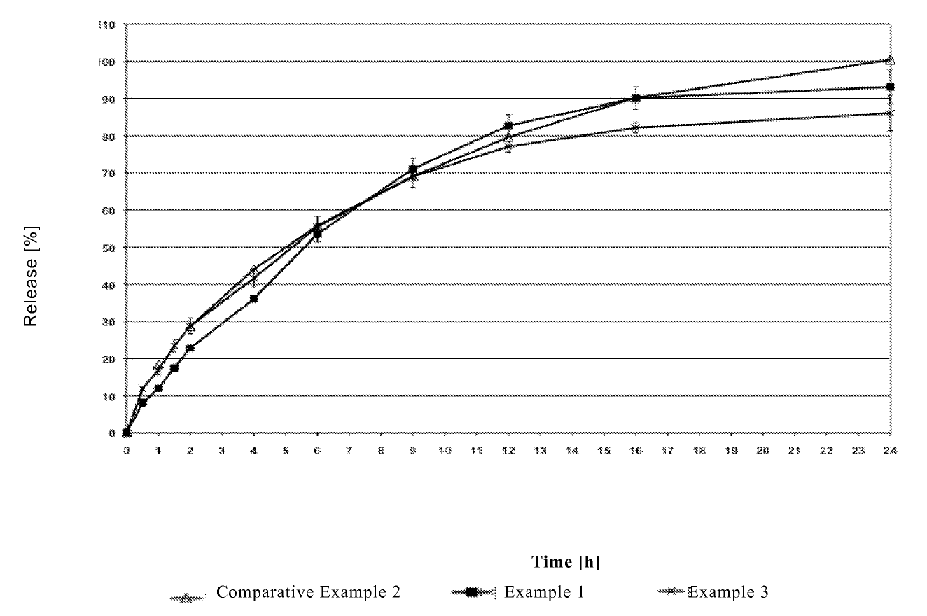

FIG. 1 shows the release profiles of comparative example 2 and Examples 1 and 3.

### EXAMPLES

### Comparative Example 1

For a better comparison with the state of the art, Example 30 of the international patent application WO 2007/052125 was repeated:

| | | Amount | Amount |
|---|---|---|---|
| Component | Function | [mg] | [%] |
| Pregabalin | active agent | 100.7 | 26.8 |
| Kollidon ® SR | matrix former | 85.3 | 22.7 |
| Plasdone XL ® | swelling agent | 93.7 | 25.0 |
| Polyox ® WSR N60K NF | swelling agent | 75.0 | 20.0 |
| Carbopol ® 71G | gelling agent | 18.7 | 5.0 |
| Magnesium stearate | lubricant | 1.9 | 0.5 |
| | Σ | 375.3 | 100.0 |

Pregabalin, Kollidon® SR, Plasdone XL®, Polyox® WSR N60K NF and Carbopol® 71G were mixed for 5 minutes in a free-fall mixer (Turbula® TB 10). The mixture obtained was screened with a screen with a mesh width of 800 μm. The screened mixture was then mixed again for 10 minutes in the free-fall mixer. Magnesium stearate was screened with a screen with a mesh width of 300 μm and added to the mixture, then it was all mixed again for 5 minutes in the free-fall mixer. After that, the mixture was compressed using an eccentric press (Korsch® EK0) with 12 mm biconvex punches and a pressing force of 24 kN to form a tablet with the following properties:

| Pressing force [kN] | Hardness [N] | Diameter [mm] | Thickness [mm] |
|---|---|---|---|
| 24 | 150 | 12.1 | 4.65 |

Measuring the swelling index revealed the swelling behaviour shown in the following table (1 L 37° C. warm 0.06 N HCl in 2,000 mL beaker, measurement of the diameter using a sliding caliper):

| Time [hours] | Diameter [mm] | % increase |
|---|---|---|
| 0 | 12.1 | |
| 1 | 17.0 | 40.5 |
| 2 | 17.0 | 40.5 |
| 4 | 16.5 | 36.4 |
| 8 | 16.5 | 36.4 |
| 24 | 17.0 | 40.5 |

The swelling to the maximum size took place within the first hour. The diameter increased by 40.5% in that time.

### Comparative Example 2

Comparative example 1 was repeated with the same amounts and process steps, with the exception of the compression conditions. The mixture in comparative example 2 was compressed with a pressing force of 20 kN into a tablet with the properties listed below.

| Pressing force [kN] | Hardness [N] | Diameter [mm] | Thickness [mm] |
|---|---|---|---|
| 20 | 110 | 11.8 | 4.4 |

Measuring the swelling index revealed the swelling behaviour shown in the following table (conditions as above):

| Time [minutes] | Diameter [mm] | % increase |
|---|---|---|
| 0 | 11.8 | |
| 5 | 19.6 | 66.0 |
| 10 | 18.0 | 52.4 |
| 30 | 15.2 | 28.7 |

This measurement shows that a maximum swelling is reached after only 5 minutes, so that the tablets swell very quickly.

The release profile of Comparative Example 2 is illustrated in FIG. 1.

### Example 1

### Matrix Tablet with Buoyancy Agent

| | | Amount | Amount |
|---|---|---|---|
| Component | Function | [mg] | [%] |
| Pregabalin | active agent | 100.7 | 22.34 |
| Kollidon ® SR | matrix former | 100.0 | 22.19 |
| Sodium hydrogen carbonate | buoyancy agent | 43.0 | 9.54 |
| Polyox ® WSR N60K NF | swelling agent | 116.0 | 25.74 |
| Carbopol ® 71G | gelling agent | 89.0 | 19.75 |
| Magnesium stearate | lubricant | 2.0 | 0.44 |
| | Σ | 450.7 | 100.0 |

Pregabalin, Kollidon® SR, sodium hydrogen carbonate, Polyox® WSR N60K NF and Carbopol® 71G were mixed for 5 minutes in a free-fall mixer (Turbula® TB 10). The mixture obtained was screened with a screen with a mesh width of 800 μm. The screened mixture was then mixed again in the free-fall mixer for 10 minutes. Magnesium stearate was screened with a screen with a mesh width of 300 μm and added to the mixture, then it was all mixed again for 5 minutes in the free-fall mixer. After that, the mixture was compressed using an eccentric press (Korsch® EK0) with 12 mm biconvex punches and a pressing force of 20 kN to form a tablet with the properties listed below:

| Pressing force [kN] | Hardness [N] | Diameter [mm] | Thickness [mm] |
|---|---|---|---|
| 20 | 192 | 12.0 | 4.8 |

With the same pressing force, the tablets of the invention have noticeably better hardness than Comparative Example 2 (110 N).

Measuring the swelling index (conditions as above) revealed the swelling behaviour shown in the following table:

| Time [hours] | Diameter [mm] | % increase |
|---|---|---|
| 0 | 12.0 | |
| 1 | 13.5 | 11.7 |
| 2 | 14.2 | 18.1 |
| 4 | 15.0 | 24.4 |
| 8 | 15.2 | 26.0 |
| 24 | 19.0 | 57.8 |

The swelling happened considerably more slowly than in the two comparative examples and took place over 24 hours.

In addition, the tablet rose within 30 seconds (1 L 37° C. warm 0.06 N HCl in a 2,000 mL beaker, measurement of the diameter using a sliding caliper).

Despite the different swelling behaviour, approximately the same release behaviour can be obtained with Example 1 as with Comparative Example 2. This can be seen by comparing the release profiles in FIG. 1.

### Example 2

### Matrix Tablet with Buoyancy Agent

Example 1 was repeated, obtaining a tablet with the properties listed below:

| Pressing force [kN] | Hardness [N] | Diameter [mm] | Thickness [mm] |
|---|---|---|---|
| 20 | 200 | 12.1 | 4.5 |

Measuring the swelling index (conditions as above) revealed the swelling behaviour shown in the following table:

| Time [minutes] | Diameter [mm] | % increase |
|---|---|---|
| 0 | 12.1 | |
| 5 | 13.2 | 9.1 |
| 10 | 13.3 | 10.8 |
| 30 | 13.5 | 11.6 |

The swelling thus happens considerably more slowly than in the comparative examples. Whereas in Comparative Example 2, the final swelling volume is in effect reached after 5 minutes, the swelling volume and thus the swelling index in Example 2 increases only slowly.

### Example 3

### Matrix Tablet with Sedimentation Agent

| | | Amount | Amount |
|---|---|---|---|
| Component | Function | [mg] | [%] |
| Pregabalin | active agent | 100.7 | 22.51 |
| Kollidon ® SR | matrix former | 99.9 | 22.33 |
| Sodium chloride | sedimentation agent | 40.0 | 8.94 |
| Polyox ® WSR N60K NF | swelling agent | 115.9 | 25.91 |
| Carbopol ® 71G | gelling agent | 88.9 | 19.87 |
| Magnesium stearate | lubricant | 1.9 | 0.42 |
| | Σ | 447.3 | 100.0 |

Pregabalin, Kollidon® SR, sodium chloride, Polyox® WSR N60K NF and Carbopol® 71G were mixed for 5 minutes in a free-fall mixer (Turbula® TB 10). The mixture obtained was screened with a screen with a mesh width of 800 μm. The screened mixture was then mixed again for 10 minutes in the free-fall mixer. Magnesium stearate was screened with a screen with a mesh width of 300 μm and added to the mixture, then it was all mixed again for 5 minutes in the free-fall mixer. After that, the mixture was compressed using an eccentric press (Korsch® EK0) with 12 mm biconvex punches and a pressing force of 21 kN to form a tablet with the following properties:

| Pressing force [kN] | Hardness [N] | Diameter [mm] | Thickness [mm] |
|---|---|---|---|
| 21 | 180 | 12 | 4.7 |

Example 3 also has noticeably better hardness than the comparative examples.

Measuring the swelling index revealed the swelling behaviour shown in the following table:

| Time [hours] | Diameter [mm] | % increase |
|---|---|---|
| 0 | 11.9 | |
| 1 | 13.7 | 15.1 |
| 2 | 15.0 | 26.4 |
| 3 | 15.2 | 27.9 |
| 4 | 15.2 | 28.5 |
| 8 | 15.9 | 34.3 |
| 24 | 17.4 | 46.5 |

The swelling happened considerably more slowly than in the comparative example and took place over 24 hours.

After being placed in 1 L 37° C. warm 0.06 N HCl in a 2,000 mL beaker, the tablet sank to the bottom of the beaker at once, so that sedimentation was immediate.

The release profile of Example 3 is illustrated in FIG. 1. Here too, despite the different swelling behaviour, approximately the same release behaviour can be achieved as with Comparative Example 2.

### Example 4

### Matrix Tablet with Sedimentation Agent

Example 3 was repeated, obtaining a tablet with the following properties:

| Pressing force [kN] | Hardness [N] | Diameter [mm] | Thickness [mm] |
|---|---|---|---|
| 20 | 180 | 12 | 4.6 |

After being placed in 1 L 37° C. warm 0.06 N HCl in a 2,000 mL beaker, this tablet, too, immediately sank to the bottom of the beaker.

Measuring the swelling index revealed the swelling behaviour in the first half hour shown in the following table:

| Time [minutes] | Diameter [mm] | % increase |
|---|---|---|
| 0 | 12.1 | |
| 5 | 12.9 | 6.6 |
| 10 | 13.2 | 9.1 |
| 30 | 13.3 | 10.8 |

1. An oral dosage form for the modified release of pregabalin, comprising pregabalin in a matrix comprising a swelling agent, a matrix former and a buoyancy agent or alternatively a sedimentation agent.

2. The oral dosage form as claimed in claim 1, wherein, after 10 minutes of swelling in deionised water at 37° C., a volume of the oral dosage form is no more than 30% greater than a volume of the dosage form before swelling.

3. The oral dosage form as claimed in claim 1, wherein the buoyancy agent is able, upon contact with 0.1 M hydrochloric acid, to release a gas.

4. The oral dosage form as claimed in claim 3, wherein the buoyancy agent is selected from the group consisting of carbonates, hydrogen carbonates, and combinations thereof, optionally together with citric acid, ascorbic acid, and/or tartaric acid.

5. The oral dosage form as claimed claim 1, wherein the sedimentation agent is selected from inorganic salts.

6. The oral dosage form as claimed in claim 1, wherein the swelling agent comprises a polyethylene glycol.

7. The oral dosage form as claimed in claim 1, wherein the matrix former comprises polyvinyl acetate, a mixture of polyvinyl acetate and polyvinyl pyrrolidone, polyethylene glycol, polyvinyl alcohol, cellulose, cellulose ether, cellulose ester, gelatine, gum arabic, carrageenan, gelatine, gum traganth, amylose, maltodextrins, polysaccharides, starch, modified starch, pectin, sugar alcohol or combinations.

8. The oral dosage form as claimed in claim 1, wherein a dimension of the oral dosage form after 30 minutes of swelling in 0.1 M hydrochloric acid at 37° C. is 9 mm or more.

9. The oral dosage form as claimed in claim 1, with a release behaviour, measured with USP Test Apparatus II using 900 ml 0.06 N HCl at pH 1.4 at a speed of 50 revolutions per minute and at a temperature of 37° C., which satisfies at least one of the following conditions:

| Time [minutes] | % pregabalin released |
|---|---|
| 30 | 5-20 |
| 60 | 10-25 |
| 90 | 15-30 |
| 120 | 20-40 |
| 320 | 30-60 |
| 560 | 50-80 |
| 720 | 60-90 |

10. The oral dosage form as claimed in claim 1 for administration once daily.

11. The oral dosage form as claimed in claim 1, comprising 50 to 600 mg pregabalin.

12. The oral dosage form as claimed in claim 1 for the prevention or treatment of a disease or complaints responsive to pregabalin.

13. The oral dosage form as claimed in claim 12, for the prevention or treatment of epilepsy, neuropathological pain, generalised anxiety disorders and/or fibromyalgia, especially neuropathological pain in connection with diabetic polyneuropathy, post-zoster neuralgia, tumours, chemotherapy, trigeminal neuralgia, alcohol abuse, vitamin B deficiency, phantom pain, borrelia infection, complex regional pain syndrome, carpal tunnel syndromes, back pain and/or AIDS.

14. A process for the production of an oral dosage form as claimed in any of the preceding claims, comprising:
(a) mixing pregabalin, matrix former, swelling agent and optionally one or more pharmaceutically acceptable excipients and subsequently compressing the resulting mixture into a tablet, or
(b) granulating pregabalin, matrix former, swelling agent and optionally one or more pharmaceutically acceptable excipients into granules, optionally adding one or more pharmaceutically acceptable excipients to the granules and subsequently compressing them into a tablet,

15. The oral dosage form as claimed in claim 3, wherein the released gas is carbon dioxide and/or nitrogen.

16. The oral dosage form as claimed claim 5, wherein the inorganic salts are chloride salts of alkali or alkaline earth.

17. The oral dosage form as claimed in claim 6, wherein the polyethylene glycol is a non-ionogenic polyethylene glycol, a cellulose derivative, polyvinyl alcohol, polyvinyl pyrrolidone, carrageenan, pectin, alginate, colloidal magnesium-aluminium silicate or a combination thereof.
